(19) United States
(12) Patent Application Publication
(10) Pub. No.: US 2026/0047822 A1
(43) Pub. Date: Feb. 19, 2026

(54) SYSTEMS AND METHODS FOR TRACKING AN OUT-OF-PLANE NEEDLE WITHIN AN ULTRASOUND IMAGE FEED

(71) Applicant: Clarius Mobile Health Corp., Vancouver (CA)

(72) Inventor: Kris Dickie, Vancouver (CA)

(21) Appl. No.: 18/807,338

(22) Filed: Aug. 16, 2024

(51) Int. Cl. A61B 8/00; A61B 8/08

(52) U.S. Cl. CPC A61B 8/463; A61B 8/0841; A61B 8/488; A61B 8/5276

(57) ABSTRACT

A method for tracking an out-of-plane needle within an ultrasound image feed that is acquired from an ultrasound scanner comprises displaying, on a screen that is communicatively connected to the ultrasound scanner, the ultrasound image feed comprising a region of interest for imaging the out-of-plane needle; activating a tissue Doppler mode of the ultrasound scanner, in which the ultrasound scanner obtains a tissue Doppler ultrasound signal corresponding to the region of interest; identifying a tissue velocity within the region of interest while the out-of-plane needle is imaged within the region of interest; and generating a visual indicator onto the ultrasound image feed to identify the imaged out-of-plane needle.

### FIELD OF THE INVENTION

The present disclosure relates generally to ultrasound imaging, and in particular, systems and methods for tracking an out-of-plane needle within an ultrasound image feed.

### BACKGROUND OF THE INVENTION

Ultrasound imaging systems are a powerful tool for performing real-time imaging procedures in a wide range of medical applications. For example, in intervention procedures (e.g., nerve blocks, vascular access), needles are often used for administration of medicine or evacuation of fluid contents. Using ultrasound guidance while inserting a needle can enhance the safety and efficacy of procedures by increasing the accuracy of the needle path to the target site.

During an interventional ultrasound procedure, a clinician may be concerned about the location and trajectory of a needle inserted into a patient. The clinician needs to understand exactly where the needle tip is located for both patient safety and clinical effectiveness. To complete a successful interventional procedure, the clinician must accurately position the needle tip in the desired anatomy while avoiding any undue tissue damage during the process of inserting and positioning the needle. In addition to avoiding particular anatomical regions, oftentimes the clinician needs to position the needle in extremely close proximity to other structures. In order to safely accomplish an interventional ultrasound procedure, the clinician needs to accurately comprehend the full path of the needle.

To assist ultrasound operators in distinguishing the needle from other imaged tissue, it is helpful to highlight or otherwise enhance the appearance of a needle in an ultrasound image feed. Traditional enhancement techniques may use ultrasound beam steering along with modification of imaging parameters like dynamic range and noise floor to enhance acoustically reflective structures. However, this may result in imaging artifacts since reflective structures perpendicular to the ultrasound beam are highlighted. Other needle highlighting techniques like magnetic tracking also provide needle trajectory information but such methods rely on additional bulky/cumbersome hardware and specialized equipment.

To truly highlight just the needle itself without the use of additional hardware, an ultrasound system must first identify an imaged needle in an ultrasound image. Traditional techniques for identifying a needle typically involve image analysis that can result in false positives because there may be tissue structures (e.g., bones, blood vessel walls) that also appear like a needle in an ultrasound image.

There is thus a need for improved ultrasound systems and methods that identify an imaged needle in an ultrasound image without the use of additional or specialized equipment. The embodiments discussed herein may address and/or ameliorate at least some of the aforementioned drawbacks identified above. The foregoing examples of the related art and limitations related thereto are intended to be illustrative and not exclusive. Other limitations of the related art will become apparent to those of skill in the art upon a reading of the specification and a study of the drawings herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Non-limiting examples of various embodiments of the present disclosure will next be described in relation to the drawings, in which:

FIG. 1 is a schematic diagram of an ultrasound imaging system, according to an embodiment of the present invention;

FIG. 2 is a schematic diagram of multiple ultrasound imaging systems of FIG. 1 that are connected to a server via a communications network, according to an embodiment of the present invention;

FIG. 3A is an illustrative perspective view of needle insertion into an anatomical structure when using an ultrasound scanner of the ultrasound imaging system of FIG. 1, during an interventional ultrasound procedure;

FIG. 3B is another illustrative perspective view of needle insertion into the anatomical structure when using the ultrasound scanner of the ultrasound imaging system of FIG. 1, during an interventional ultrasound procedure;

FIG. 4A is a flowchart diagram showing acts of a method for tracking an out-of-plane needle within an ultrasound image feed that is acquired from an ultrasound scanner, in accordance with at least one embodiment of the present invention;

FIG. 4B is a flowchart diagram showing acts of another method for tracking an out-of-plane needle within an ultrasound image feed that is acquired from an ultrasound scanner, in accordance with at least one embodiment of the present invention;

FIG. 5A is a diagram showing the illustrative perspective view of FIG. 3B and an exemplary graphical user interface displayed to a user during the interventional ultrasound procedure, in accordance with at least one embodiment of the present invention;

FIG. 5B is a diagram showing the illustrative perspective view of FIG. 3B and another exemplary graphical user interface displayed to a user during the interventional ultrasound procedure, in accordance with at least one embodiment of the present invention;

FIG. 5C is an exemplary B-mode ultrasound image of a human anatomical structure that is generated during implementation of the method of FIG. 4A (or FIG. 4B), in accordance with at least one embodiment of the present invention;

FIG. 5D, for comparison, is a color Doppler image of the human anatomical structure of FIG. 5C, showing typical blood flow without the application of tissue Doppler;

FIG. 5E is an exemplary tissue Doppler mode image of the human anatomical structure of FIG. 5C, that is generated during implementation of the method of FIG. 4A (or FIG. 4B) and showing wall motion, in accordance with at least one embodiment of the present invention;

FIG. 6A is an exemplary B-mode ultrasound image generated during implementation of the method of FIG. 4A (or FIG. 4B), in accordance with at least one embodiment of the present invention;

FIG. 6B is an exemplary tissue Doppler mode ultrasound image generated during implementation of the method of FIG. 4A (or FIG. 4B), in accordance with at least one embodiment of the present invention;

FIG. 6C is another exemplary tissue Doppler mode ultrasound image generated during implementation of the method of FIG. 4A (or FIG. 4B), in accordance with at least one embodiment of the present invention;

FIG. 6D is another exemplary B-mode ultrasound image generated during implementation of the method of FIG. 4A (or FIG. 4B), in accordance with at least one embodiment of the present invention;

FIG. 6E is another exemplary tissue Doppler mode ultrasound image generated during implementation of the method of FIG. 4A (or FIG. 4B), in accordance with at least one embodiment of the present invention;

FIG. 6F is an image showing the application of a sample/color box result on an example ultrasound image, in accordance with at least one embodiment of the present invention;

FIG. 6G is an image showing the application of tissue Doppler on the image of FIG. 6F, in accordance with at least one embodiment of the present invention;

FIG. 6H is an image showing the result of performing acts of adjusting opacity, on an example ultrasound image, in accordance with at least one embodiment of the present invention;

FIG. 6I is an additional image showing the result of performing acts of adjusting opacity, on an example ultrasound image, in accordance with at least one embodiment of the present invention;

FIG. 6J is an additional image showing the result of performing acts of adjusting opacity, on an example ultrasound image, in accordance with at least one embodiment of the present invention;

FIG. 7A is an exemplary tissue Doppler mode ultrasound image showing image artefacts that can be generated during implementation of the method of FIG. 4A (or FIG. 4B), in accordance with at least one embodiment of the present invention;

FIG. 7B is another exemplary tissue Doppler mode ultrasound image showing image artefacts that can be generated during implementation of the method of FIG. 4A (or FIG. 4B), in accordance with at least one embodiment of the present invention;

FIG. 8A is a touchscreen user interface showing a B-mode image, comprising a region of interest with a vessel, along with drop-down screen mode options, according to an embodiment of the present invention;

FIG. 8B is a touchscreen as shown in FIG. 8A, in color Doppler mode, comprising a sample box/color box, according to an embodiment of the present invention; and

FIG. 8C is a touchscreen as shown in FIG. 8B, in tissue Doppler mode/tissue Doppler imaging (TDI), according to an embodiment of the present invention.

Unless otherwise specifically noted, articles depicted in the drawings are not necessarily drawn to scale.

### DETAILED DESCRIPTION OF THE INVENTION

### A. Glossary

The term “communications network” and “network” can include both a mobile network and data network without limiting the term's meaning, and includes the use of wireless (e.g. 2G, 3G, 4G, 5G, WiFi®, WiMAX®, Wireless USB (Universal Serial Bus), Zigbee®, Bluetooth® and satellite), and/or hard wired connections such as local, internet, ADSL (Asymmetrical Digital Subscriber Line), DSL (Digital Subscriber Line), cable modem, T1, T3, fiber-optic, dial-up modem, television cable, and may include connections to flash memory data cards and/or USB memory sticks where appropriate. A communications network could also mean dedicated connections between computing devices and electronic components, such as buses for intra-chip communications.

The term “module” can refer to any component in this invention and to any or all of the features of the invention without limitation. A module may be a software, firmware or hardware module (or part thereof), and may be located or operated within, for example, in the ultrasound scanner, a display device or a server.

The term “multi-purpose electronic device” or “display device” or “computing device” or “off-the-shelf display computing device” or “mobile device” is intended to have broad meaning and includes devices with a processor communicatively operable with a screen interface, for example, such as, laptop computer, a tablet computer, a desktop computer, a smart phone, a smart watch, spectacles with a built-in display, a television, a bespoke display or any other display device that is capable of being communicably connected to an ultrasound scanner. Such a device may be communicatively operable with an ultrasound scanner and/or a cloud-based server (for example via one or more communications networks). Such device may be combined with processor, non-transitory memory, and/or user input device in a shared electronic device, or there may be peripheral display devices which may comprise a monitor, touchscreen, projector, or other display device known in the art, which may enable a user to view ultrasound images produced by an ultrasound imaging system, and/or interact with various data stored in non-transitory memory.

The term “operator” (or “user”) may (without limitation) refer to the person that is operating an ultrasound scanner (for example, a clinician, medical personnel, a sonographer trainer, a student, a vet, a sonographer/ultrasonographer and/or ultrasound technician). This list is non-exhaustive.

The term “processor” can refer to any electronic circuit or group of circuits that perform calculations, and may include, for example, single or multicore processors, multiple processors, an ASIC (Application Specific Integrated Circuit), and dedicated circuits implemented, for example, on a reconfigurable device such as an FPGA (Field Programmable Gate Array). A processor may perform the steps in the flowcharts and sequence diagrams, whether they are explicitly described as being executed by the processor or whether the execution thereby is implicit due to the steps being described as performed by the system, a device, code or a module. The processor, if comprised of multiple processors, may be located together or geographically separate from each other. The term includes virtual processors and machine instances as in cloud computing or local virtualization, which are ultimately grounded in physical processors.

The term “scan convert”, “scan conversion”, or any of its grammatical forms refers to the construction of an ultrasound media, such as a still image or a video, from lines of ultrasound scan data representing echoes of ultrasound signals. Scan conversion may involve converting beams and/or vectors of acoustic scan data which are in polar (R-theta) coordinates to cartesian (X-Y) coordinates.

The term “system” when used herein, and not otherwise qualified, may include an ultrasound scanner and a multi-purpose electronic device/display device; and/or an ultrasound scanner, multi-purpose electronic device/display device and a server. The system may include one or more applications operating on a multi-purpose electronic device/display device to which the ultrasound scanner is communicatively connected.

The term “ultrasound image frame” (or “image frame” or “ultrasound frame”) refers to a frame of either pre-scan data or post-scan conversion data that is suitable for rendering an ultrasound image on a screen or other display device.

The term “ultrasound transducer” (or “probe” or “ultrasound probe” or “transducer” or “ultrasound scanner” or “scanner” or “ultrasound imaging device”) refers to a wide variety of transducer types including but not limited to linear transducer, curved transducers, curvilinear transducers, convex transducers, microconvex transducers, and endocavity probes. In operation, an ultrasound scanner is often communicatively connected to a multi-purpose electronic device/display device to direct operations of the ultrasound scanner, optionally through one or more applications on the multi-purpose electronic device/display device (for example, via the Clarius™ App).

The term “workflow application” or “application” (for example, via the Clarius™ App) or “workflow” refers to a software tool that assists with the automated activation and/or configuration of device feature(s). Conveyance to an operator may be visually on the display screen or via audio.

### B. Exemplary Embodiments

In a first broad aspect of the present disclosure, there are provided ultrasound imaging systems and method for tracking an out-of-plane needle within an ultrasound image feed.

In another broad aspect of the present disclosure, there is provided a method for tracking an out-of-plane needle within an ultrasound image feed that is acquired from an ultrasound scanner. The method includes: displaying, on a screen that is communicatively connected to the ultrasound scanner, the ultrasound image feed including a region of interest for imaging the out-of-plane needle; activating a tissue Doppler mode of the ultrasound scanner, in which the ultrasound scanner obtains a tissue Doppler ultrasound signal corresponding to the region of interest; identifying a tissue velocity within the region of interest while the out-of-plane needle is imaged within the region of interest; and generating a visual indicator onto the ultrasound image feed to identify the imaged out-of-plane needle.

In another broad aspect of the present disclosure, there is provided an ultrasound imaging system for tracking an out-of-plane needle within an ultrasound image feed. The ultrasound imaging system includes an ultrasound scanner, a processor that is communicatively connected to the ultrasound scanner, and a display device. The ultrasound scanner is configured to acquire a plurality of new ultrasound image frames. The processor is configured to: display, on a screen that is communicatively connected to the ultrasound scanner, the ultrasound image feed including a region of interest for imaging the out-of-plane needle; activate a tissue Doppler mode of the ultrasound scanner, in which the ultrasound scanner obtains a tissue Doppler ultrasound signal corresponding to the region of interest; identify a tissue velocity within the region of interest while the out-of-plane needle is imaged within the region of interest; and generate a visual indicator on the ultrasound image feed to identify the imaged out-of-plane needle. The display device is configured to display at least the visual indicator to a system user.

In another broad aspect of the present disclosure, there is provided a computer readable medium storing instructions for execution by a processor communicatively coupled with an ultrasound scanner, within an ultrasound imaging system. When the instructions are executed by the processor, it is configured to: display, on a screen that is communicatively connected to the ultrasound scanner, the ultrasound image feed including a region of interest for imaging the out-of-plane needle; activate a tissue Doppler mode of the ultrasound scanner, in which the ultrasound scanner obtains a tissue Doppler ultrasound signal corresponding to the region of interest; identify a tissue velocity within the region of interest while the out-of-plane needle is imaged within the region of interest; and generate a visual indicator on the ultrasound image feed to identify the imaged out-of-plane needle.

For simplicity and clarity of illustration, where considered appropriate, reference numerals may be repeated among the figures to indicate corresponding or analogous elements or steps. In addition, numerous specific details are set forth in order to provide a thorough understanding of the exemplary embodiments described herein. However, it will be understood by those of ordinary skill in the art that the embodiments described herein may be practiced without these specific details. In other instances, certain steps, signals, protocols, software, hardware, networking infrastructure, circuits, structures, techniques, well-known methods, procedures and components have not been described or shown in detail in order not to obscure the embodiments generally described herein.

Furthermore, this description is not to be considered as limiting the scope of the embodiments described herein in any way. It should be understood that the detailed description, while indicating specific embodiments, are given by way of illustration only, since various changes and modifications within the scope of the disclosure will become apparent to those skilled in the art from this detailed description. Accordingly, the specification and drawings are to be regarded in an illustrative, rather than a restrictive, sense.

The system of the present invention uses a transducer (e.g., a piezoelectric or capacitive device operable to convert between acoustic and electrical energy) to scan a planar region or a volume of an anatomical structure. Electrical and/or mechanical steering allows transmission and reception along different scan lines wherein any scan pattern may be used. Ultrasound data representing a plane or volume is provided in response to the scanning. The ultrasound data is beamformed, detected, and/or scan converted. The ultrasound data may be in any format, such as polar coordinate, Cartesian coordinate, a three-dimensional grid, two-dimensional planes in Cartesian coordinate with polar coordinate spacing between planes, or other format. The ultrasound data is data which represents an anatomical structure sought to be assessed and reviewed by a sonographer.

A user input device may comprise one or more of a touchscreen, a keyboard, a mouse, a trackpad, a motion sensing camera, or other device configured to enable a user to interact with and manipulate data within an image processing system. A display device may include one or more display devices utilizing virtually any type of technology. In some embodiments, display device may be part of a multi-purpose display device or may comprise a computer monitor, and in both cases, may display ultrasound images. A display device may be combined with processor, non-transitory memory, and/or user input device in a shared electronic device, or there may be peripheral display devices which may comprise a monitor, touchscreen, projector, or other display device known in the art, which may enable a user to view ultrasound images produced by an ultrasound imaging system, and/or interact with various data stored in non-transitory memory.

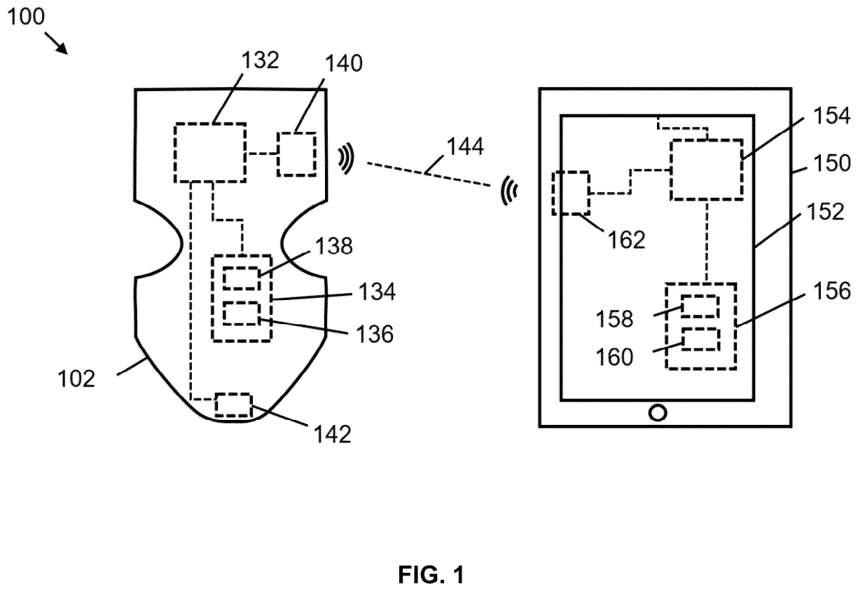

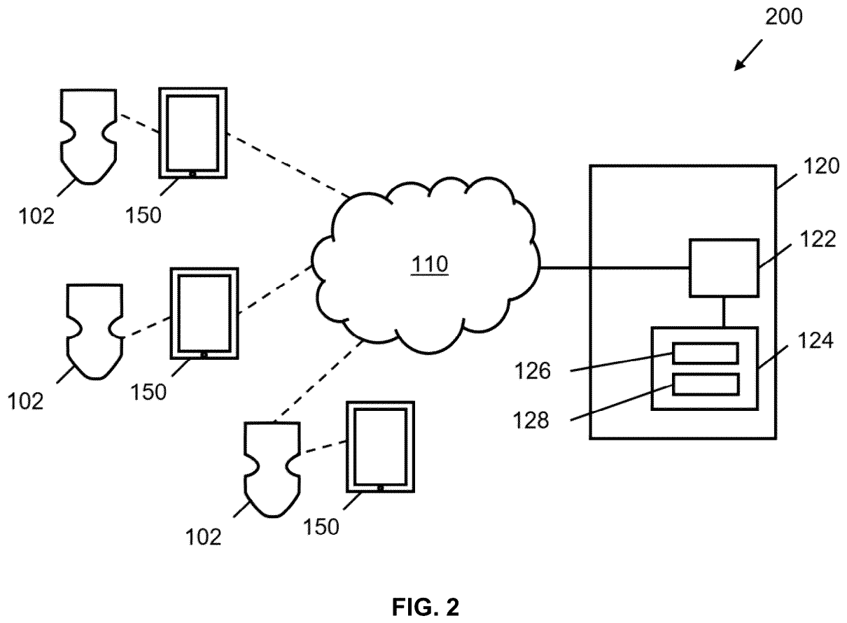

Reference will now be made to FIGS. 1 and 2. In FIG. 1, there is shown an exemplary ultrasound imaging system 100 for tracking an out-of-plane needle within an ultrasound image feed. The system 100 includes an ultrasound scanner 102 with a processor 132, which is connected to a non-transitory computer readable memory 134 storing computer readable instructions 136, which, when executed by the processor 132, may cause the scanner 102 to provide one or more of the functions of the system 100. Such functions may be, for example, the acquisition of ultrasound data, the processing of ultrasound data, the scan conversion of ultrasound data, the transmission of ultrasound data or ultrasound frames to a display device 150, the detection of operator inputs to the ultrasound scanner 102, and/or the switching of the settings of the ultrasound scanner 102.

Also stored in the computer readable memory 134 may be computer readable data 138, which may be used by the processor 132 in conjunction with the computer readable instructions 136 to provide the functions of the system 100. Computer readable data 138 may include, for example, configuration settings for the scanner 102, such as presets that instruct the processor 132 how to collect and process the ultrasound data for a plurality of regions of interest (ROIs) and how to acquire a series of ultrasound frames.

The scanner 102 may include an ultrasonic transducer 142 that transmits and receives ultrasound energy in order to acquire ultrasound frames. The scanner 102 may include a communications module 140 connected to the processor 132. In the illustrated example, the communications module 140 may wirelessly transmit signals to and receive signals from the display device 150 along wireless communication link 144. The protocol used for communications between the scanner 102 and the display device 150 may be WiFi™ or Bluetooth™, for example, or any other suitable two-way radio communications protocol. In some embodiments, the scanner 102 may operate as a WiFi™ hotspot, for example. Communication link 144 may use any suitable wireless communications network connection. In some embodiments, the communication link 144 between the scanner 102 and the display device 150 may be wired. For example, the scanner 102 may be attached to a cord that may be pluggable into a physical port of the display device 150.

The display device 150 can include any multi-purpose electronic devices that can host a screen 152, and may include a processor 154, which may be connected to a non-transitory computer readable memory 156 storing computer readable instructions 158, which, when executed by the processor 154, cause the display device 150 to provide one or more of the functions of the system 100. Such functions may be, for example, the receiving of ultrasound data that may or may not be pre-processed; scan conversion of received ultrasound data into an ultrasound image; processing of ultrasound data in image data frames; the display of a user interface; the control of a probe and the display of an ultrasound image feed on the screen to, for example, identify one or more dimensions of an anatomical structure and/or track the location and trajectory of an imaged needle.

In various embodiments, the display device 150 may be, for example, a laptop computer, a tablet computer, a desktop computer, a smart phone, a smart watch, spectacles with a built-in display, a television, a bespoke display or any other display device that is capable of being communicably connected to the scanner 102. The screen 152 may comprise a touch-sensitive display (e.g., touchscreen) that can detect a presence of a touch from the operator on screen 152 and can also identify a location of the touch in screen 152. The touch may be applied by, for example, at least one of an individual's hand, glove, stylus, or the like. As such, the touch-sensitive display may be used for example to toggle text or to provide other inputs. The screen 152 and/or any other user interface may also communicate audibly. The display device 150 is configured to present information to the operator during or after the imaging or data acquiring session. The information presented may include ultrasound images (e.g., one or more 2D frames), graphical elements, measurement graphics of the displayed images, user-selectable elements, user settings, and other information (e.g., administrative information, personal information of the patient, and the like).

Also stored in the computer readable memory 156 may be computer readable data 160, which may be used by the processor 154 in conjunction with the computer readable instructions 158 to provide the functions of the system 100. Computer readable data 160 may include, for example, settings for the scanner 102, such as presets for acquiring ultrasound data and/or settings for a user interface displayed on the screen 152. Settings may also include any other data that is specific to the way that the scanner 131 operates or that the display device 150 operates. It can therefore be understood that the computer readable instructions and data used for controlling the system 100 may be located either in the computer readable memory 134 of the scanner 102, the computer readable memory 156 of the display device 150, and/or both the computer readable memories 134, 156.

The display device 150 may also include a communications module 162 connected to the processor 154 for facilitating communication with the scanner 102. In the illustrated example, the communications module 162 wirelessly transmits signals to and receives signals from the scanner 102 on wireless communication link 144. However, as noted, in some embodiments, the connection between scanner 102 and display device 150 may be wired.

Such a screen may comprise a touch-sensitive display (e.g., touchscreen) that can detect a presence of a touch from the operator on screen and can also identify a location of the touch in screen. The touch may be applied by, for example, at least one of an individual's hand, glove, stylus, or the like. As such, the touch-sensitive display may be used to receive an input, for example, indicating the presence or absence of text or annotations on an image. The screen and/or any other user interface may also communicate audibly. The display device 150 may be configured to present information to the operator during or after the imaging or data acquiring session. The information presented may include ultrasound images (e.g., one or more 2D frames), graphical elements, measurement graphics of the displayed images, user-selectable elements, user settings, and other information (e.g., administrative information, personal information of the patient, and the like).

Referring to FIG. 2, a system 200 is shown in which there are multiple similar or different scanners 102 connected to their corresponding display devices 150 and either connected directly, or indirectly via the display devices, to a communications network 110, such as the internet.

The scanners 102 may be connected via the communications network 110 to a server 120. The server 120 may include a processor 122, which may be connected to a non-transitory computer readable memory 124 storing computer readable instructions 126, which, when executed by the processor 122, cause the server 120 to provide one or more of the functions of the system 100. Such functions may be, for example, the receiving of ultrasound frames, the processing of ultrasound data in ultrasound frames, the control of the scanners 102, the processing of new ultrasound images to, for example, identify one or more dimensions of an anatomical structure and/or track the location and trajectory of an imaged needle.

Also stored in the computer readable memory 124 may be computer readable data 128, which may be used by the processor 122 in conjunction with the computer readable instructions 126 to provide the functions of the system 100. Computer readable data 128 may include, for example, settings for the scanners 102 such as preset parameters for acquiring ultrasound data, and settings for user interfaces displayed on the display devices 102. Settings may also include any other data that is specific to the way that the scanners 102 operate or that the display devices 150 operate.

It can therefore be understood that the computer readable instructions and data used for controlling the system 100 may be located either in the computer readable memory of the scanners 120, the computer readable memory of the display devices 150, the computer readable memory 124 of the server 120, or any combination of the foregoing locations.

Ultrasound imaging systems (e.g., systems 100 and 200 shown in FIGS. 1 and 2 respectively) may generally be operated in various Doppler modes that take advantage of the fact that reflected echoes undergo a change in frequency when reflected by moving objects in tissue (e.g., blood in vascular tissue, a needle during an interventional ultrasound procedure). Some Doppler modes include spectral Doppler, pulsed wave (PW) Doppler, continuous wave (CW) Doppler, color Doppler, and Power Doppler. Tissue Doppler Imaging (TDI) is also a particular way of using spectral or Color Doppler for visualizing tissue wall motion using a lower frequency signal acquisition rate. It can be interchanged with the use of Power Doppler and Color Doppler as necessary.

Color Doppler produces a color-coded map of Doppler shifts superimposed onto a B-mode ultrasound image. Blood flow direction depends on whether the motion is toward or away from the transducer. Selected by convention, certain colors can provide information about the direction and velocity of the blood flow i.e., red is accepted to mean there is flow towards the ultrasound probe and blue is accepted to mean that there is flow away from the ultrasound probe. It is possible for different colors to be used, or different manners of illustration of direction on the image.

When an ultrasound scanner is used in a power Doppler mode, it allows the operator to select a specific, small area on the image, and, in the tissue corresponding to that area, measure blood motion velocity. As part of this process, a gate is specified by the user, along an ultrasound beam line or direction (e.g., a one-dimensional signal is obtained). Color doppler provides information about the presence or absence of flow, mean flow velocity and direction of flow within a selected color box on an anatomical feature. Spectral Doppler differs from Color Doppler imaging in that information is not obtained from the entire color box (as placed) but from a specified gate window, as noted above, a generally 2-4 mm wide sample volume. In power Doppler the magnitude of the color flow output is displayed rather than the Doppler frequency signal. Power Doppler does not display flow direction or different velocities, so it is often used in conjunction with frame averaging to increase sensitivity to low flows and velocities.

Pulsed wave (PW) Doppler is used for determining frequency shifts within a specific area of interest. B-mode imaging is used to set the location of a sample volume or “gate” for interrogation with pulsed wave Doppler. The ultrasound imaging device then transmits short sound pulses and waits for the returning echo. Since the speed of sound in soft tissues is known, the ultrasound imaging device can vary the period during which it is ‘listening’ for returned echoes based on the expected time the sound will need to travel to return to the transducer. PW Doppler can then isolate frequency shifts from the sample volume, which is displayed in graphical form with frequency on the y-axis and time on the x-axis. If the user inputs data regarding the Doppler angle, the machine can calculate velocity and display this in place of frequency. PW Doppler waveforms typically appear “carved out” in that they only display a narrow band of velocities. This is possible because the interrogation gate can select small areas where flow velocities only exist within a narrow range.

Color flow Doppler ultrasound produces a color-coded map of Doppler shifts superimposed onto a B-mode ultrasound image (color flow maps). Although color flow imaging uses pulsed wave ultrasound, its processing differs from that used to provide the Doppler sonogram. Color flow imaging may have to produce several thousand color points of flow information for each frame superimposed on the B-mode image. Color flow imaging uses fewer, shorter pulses along each color scan line of the image to give a mean frequency shift and a variance at each small area of measurement. This frequency shift is displayed as a color pixel. The scanner then repeats this for several lines to build up the color image, which is superimposed onto the B-mode image. The transducer elements are switched rapidly between B-mode and color flow imaging to give an impression of a combined simultaneous image. The pulses used for color flow imaging are typically three to four times longer than those for the B-mode image, with a corresponding loss of axial resolution. Assignment of color to frequency shifts is usually based on direction (for example, red for Doppler shifts towards the ultrasound beam and blue for shifts away from it) and magnitude (different color hues or lighter saturation for higher frequency shifts). The color Doppler image is dependent on general Doppler factors, particularly the need for a good beam/flow angle. Curvilinear and phased array transducers have a radiating pattern of ultrasound beams that can produce complex color flow images, depending on the orientation of the arteries and veins. In practice, the experienced operator can alter the scanning approach to obtain good insonation angles so as to achieve unambiguous flow images.

Color Doppler ultrasound uses the same principles as pulsed wave Doppler. Within a region of interest (ROI, for example a color Doppler box) many different “sample volumes or pixel areas” are assessed for calculating the velocity and direction of flow (for each individual area). This information is then encoded in color according to a color map scheme (which can be chosen by the operator) and displayed for each imaging frame (dynamic color flow imaging).

During an interventional ultrasound procedure, a clinician may be concerned about the location and trajectory of a needle inserted into a patient. The clinician needs to clearly understand exactly where the needle tip is located for both patient safety and clinical effectiveness. In order to complete a successful interventional procedure, the clinician must accurately position the needle tip in the desired anatomy while avoiding causing any undue tissue damage during the process of inserting and positioning the needle. In addition to avoiding particular anatomical regions, oftentimes the clinician is trying to position the needle in extremely close proximity to other structures. In order to safely accomplish an interventional ultrasound procedure, the clinician needs to accurately comprehend the full path of the needle.

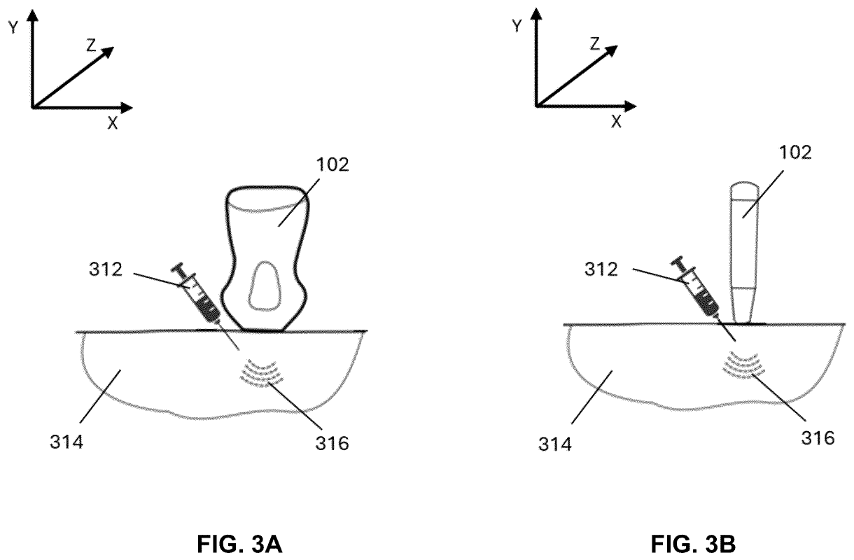

There are a variety of techniques known and employed in the art for tracking an “in-plane” needle during an interventional ultrasound procedure. Without limiting the generality of the foregoing, and by way of example, the teachings of the following are incorporated herein by reference: U.S. Pat. No. 10,102,452. However, there can be challenges associated with locating and/or tracking an out-of-plane needle during an interventional ultrasound procedure, for example, when a needle is introduced perpendicular to the plane of the ultrasound beam (see FIGS. 3A and 3B for distinction). Typically, vessels are viewed in cross section, enabling adjacent structures to be easily visualized, but this approach gives poorer visualization of the needle because, first, the angle of approach of the needle is more parallel to the ultrasound beam, and second, only one short segment of the needle is visible. What is thought to be the tip of the needle may actually be the needle shaft. This is not an ideal situation for a clinician. Herein, it has been found, specifically that the employment of tissue Doppler mode enables the tracking of out of plane needles within a region of interest being scanned, thereby providing useful information to a clinician during interventional procedures.

The embodiments of the present invention use tissue Doppler to show movement of tissue surrounding a needle (being inserted or retracted) as visual indicator or cue as to the location and movement of the out of plane needle. Tissue Doppler imaging allows for measurements of tissue movement surrounding the out of plane needle. This is possible due to the fundamental operations of tissue Doppler, which differs from traditional pulsed wave Doppler, by setting filters that discard the low amplitude/higher Doppler shift signals and instead highlight higher amplitude/lower Doppler shift signals.

Within the scope of the present invention, tissue Doppler can be performed in a variety of modes, although pulsed-wave and color modes are preferred. With color tissue Doppler (color TDI), a color-coded representation of tissue movement and/or velocity may be superimposed on gray-scale, 2-dimensional (B-mode) or M-mode images to indicate the direction and velocity of such tissue motion, in response to needle passage therethrough. Although pulsed wave may also be employed within the embodiments of the invention, color tissue Doppler mode has an advantage of increased spatial resolution and the ability to evaluate multiple structures and segments in a single view, in an imaging frame.

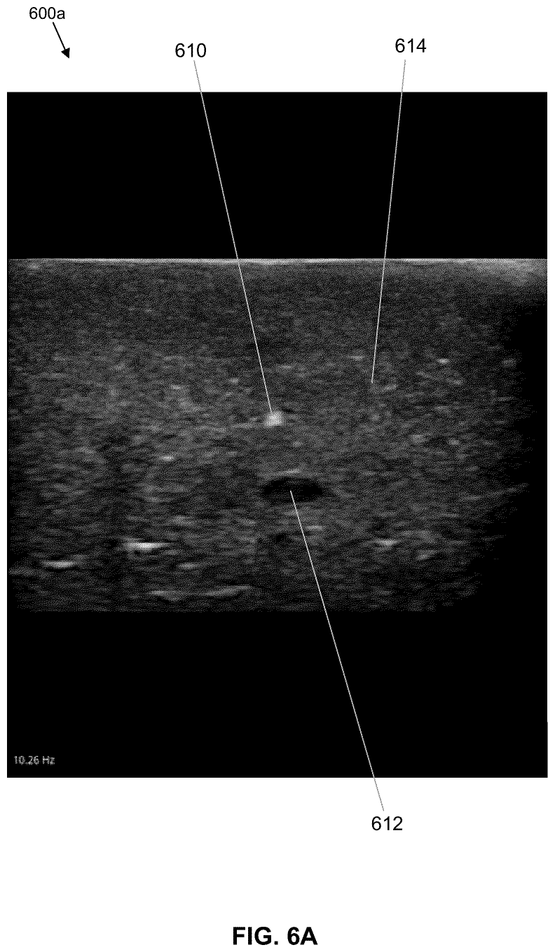

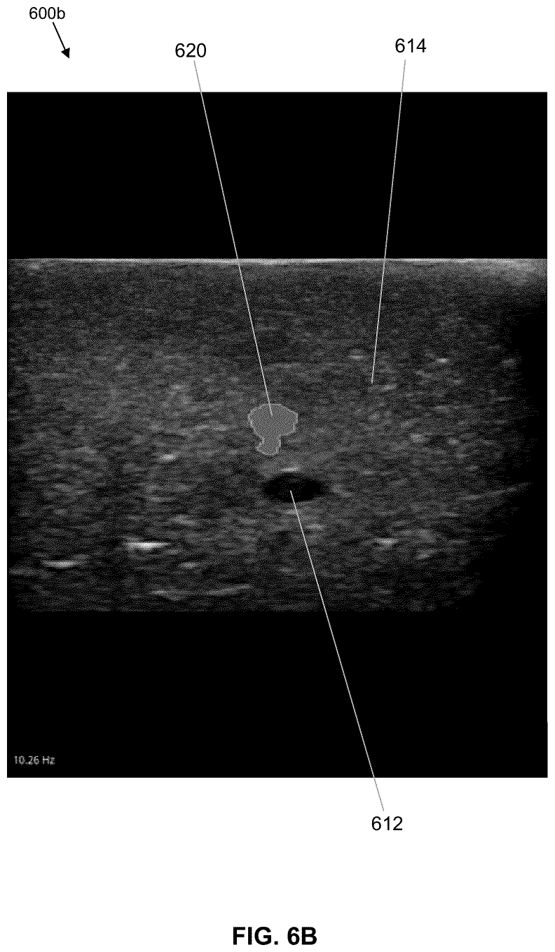

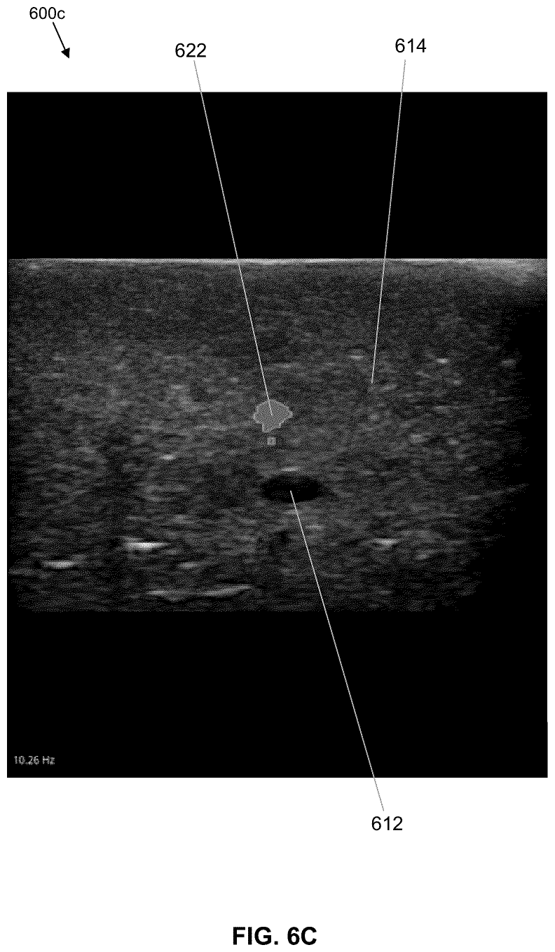

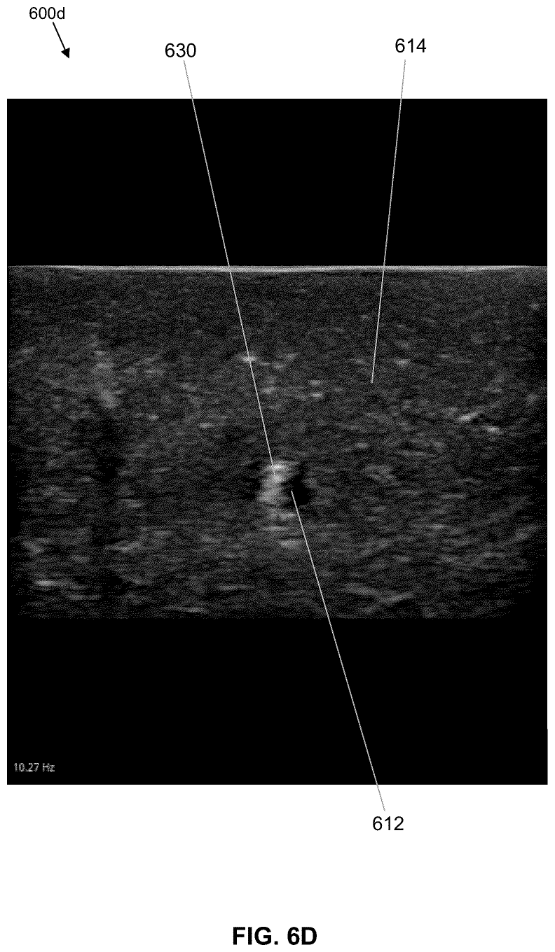

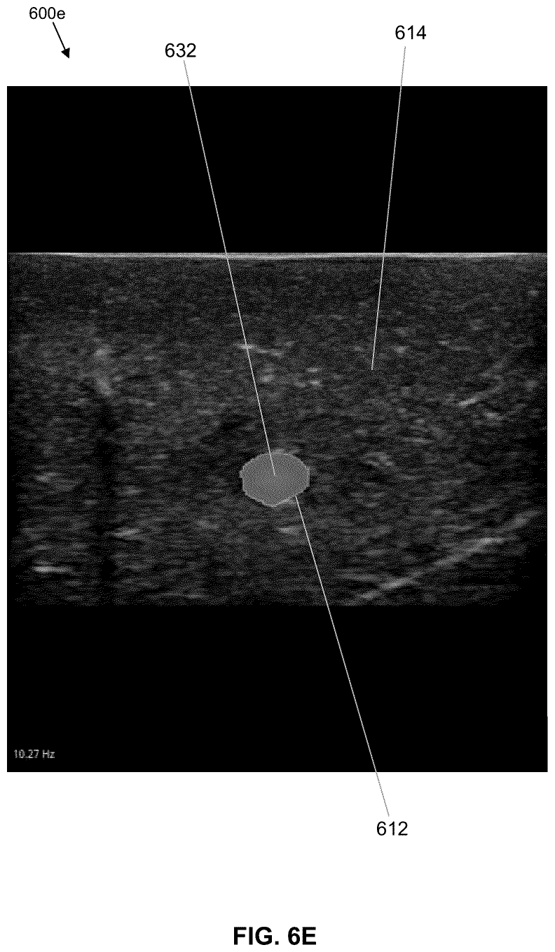

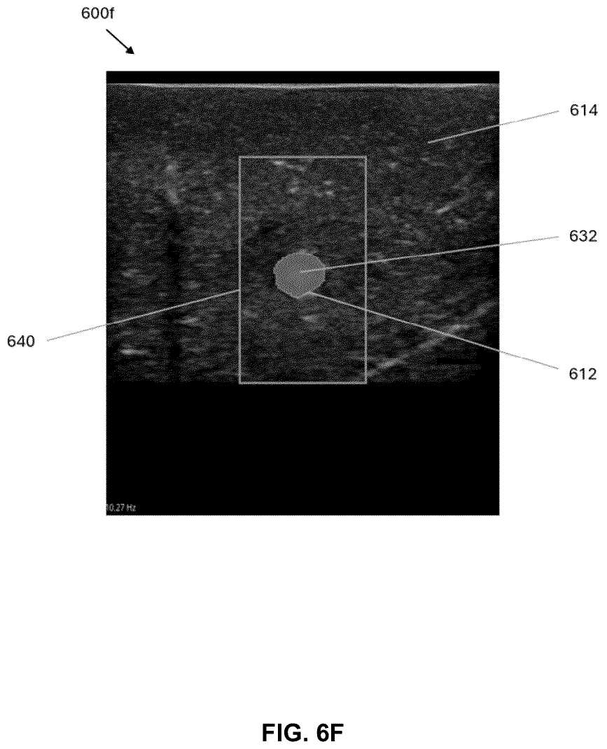

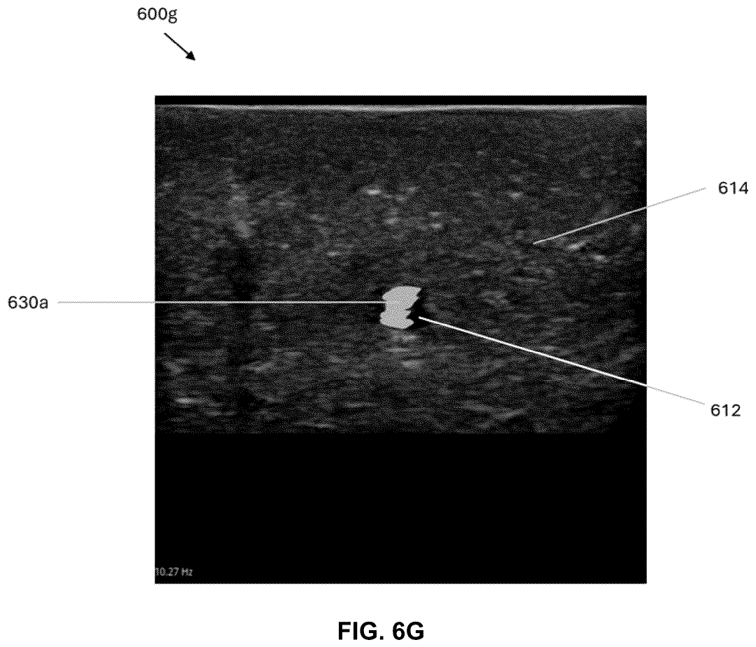

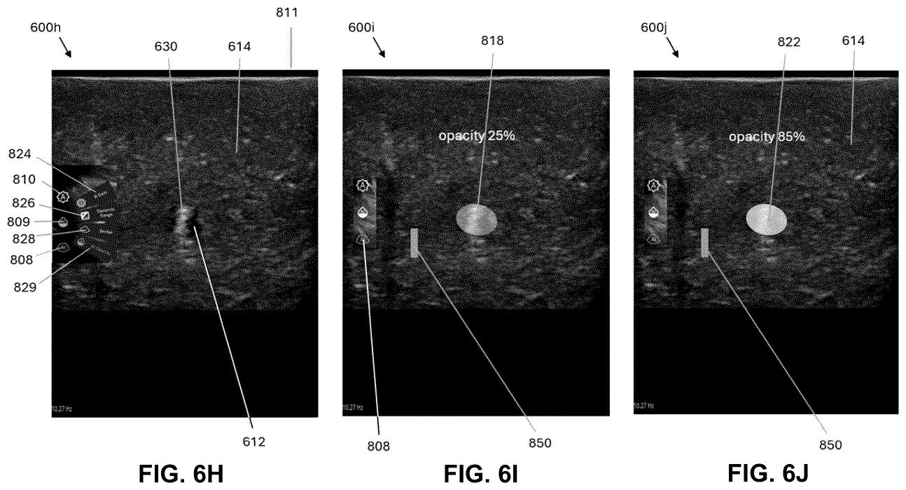

After acquisition of a B-mode image, comprising a region of interest, and into which an out of plane needle is sought to be inserted or retracted, a color box is placed on the ultrasound image in one or more of pulsed wave mode, color mode or tissue Doppler mode. Activation of tissue Doppler mode is facilitated with this box (or sample ROI) in place. When a needle is moving in that plane, it will be detected by tissue Doppler in the ultrasound image frame by, for example a color indicator or other visual cue. In some embodiments, different colors may be selected to indicate needle insertion as compared to needle retraction. Opacity of an ultrasound image can be adjusted for best view, as described below in FIGS. 6H, 6I and 6J.

Reference will now be made to FIGS. 3A and 3B showing illustrative perspective views of needle insertion into an anatomical structure 314 when using the ultrasound scanner 102 (shown previously in FIGS. 1 and 2) during an interventional ultrasound procedure. In FIG. 3A, needle 312 is introduced (or retracted) parallel to a scanning plane (e.g., an XY plane in the illustrated example) of an ultrasound beam 316 of the ultrasound scanner 102. In such examples, needle 312 may be referred to as in-plane needle 312. In FIG. 3B, needle 312 is introduced (or retracted) perpendicular to the scanning plane (e.g., a YZ plane in the illustrated example) of the ultrasound beam 316 of the ultrasound scanner 102. In such examples, needle 312 may be referred to as out-of-plane needle 312. In the example illustrated in FIG. 3B, the needle 312 is introduced in a plane XY perpendicular to the scanning plane YZ. In other examples, out-of-plane needle 312 may be introduced (or retracted) in any oblique/non-parallel plane with reference to the scanning plane.

During an interventional ultrasound procedure, various anatomical structures (e.g., blood vessels, nerves) are typically viewed in cross section, enabling adjacent structures to be easily visualized. But this approach may provide a poorer visualization of an out-of-plane needle (compared with an in-plane needle) because, first, the angle of approach of the out-of-plane needle can be more parallel (compared with an in-plane needle) to the ultrasound beam, and second, only a short segment of the out-of-plane needle may be visible in the ultrasound image. For example, this may result in a clinician misinterpreting a shaft portion of the out-of-plane needle as the tip portion of the out-of-plane needle.

The systems and methods of the present invention can enable accurate tracking of an out-of-plane needle by using a tissue Doppler mode to detect and show movement of tissues surrounding the out-of-plane needle. Tissue Doppler mode differs from a traditional pulsed wave Doppler mode by setting filters that discard the low amplitude/higher Doppler shift signals and highlight higher amplitude/lower Doppler shift signals. The tissue Doppler mode can therefore use various filters to focus on velocity of the tissue movement instead of blood movement. Tissue Doppler mode imaging is typically performed on the myocardium, for example, to assess health, elasticity, movement of cardiac walls.

The systems and methods of the present invention can use the tissue Doppler mode to detect tissue movements caused by interaction with an out-of-plane needle and use the detected tissue movements to visually indicate the spatial location and trajectory (insertion/retraction) of the out-of-plane needle. The disclosed systems and methods can generate a visual indicator onto an ultrasound image feed to identify the imaged out-of-plane needle. In some embodiments, the generated visual indicator may be overlaid onto an ultrasound image feed with an overlay position of the visual indicator identifying the location of an imaged out-of-plane needle within a region of interest. In some embodiments, a color associated with the generated visual indicator may identify an insertion or retraction direction of the imaged out-of-plane needle.

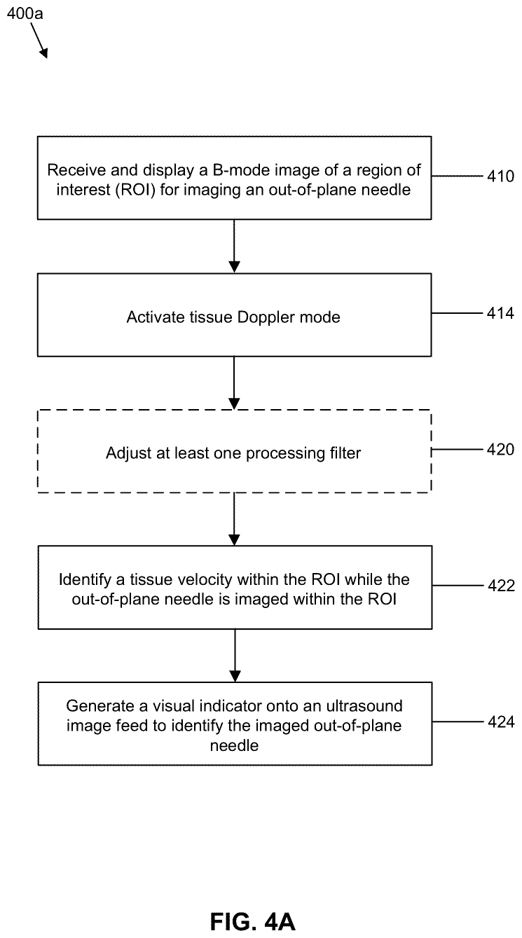

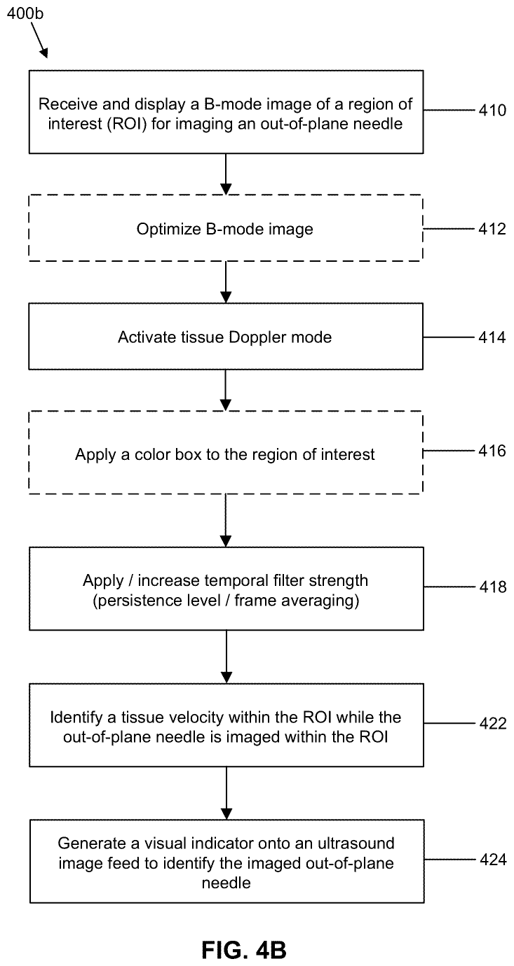

Reference will now be made to FIG. 4A. FIG. 4A is a flowchart diagram showing acts of a method 400a for tracking an out-of-plane needle within an ultrasound image feed that is acquired from an ultrasound scanner. Method 400a may generally be performed using any suitable ultrasound imaging system. For example, method 400a may be performed using ultrasound imaging system 100 shown in FIG. 1 or ultrasound imaging system 200 shown in FIG. 2 and concurrent reference is made herein below to the system components shown in FIGS. 1 and 2.

At act 410, the method 400a includes receiving and displaying, on a display device, one or more B-mode images of a region of interest for imaging the out-of-plane needle. For example, ultrasound scanner 102 may acquire a plurality of new ultrasound image frames and provide the acquired ultrasound image feed to a processor (e.g., processor 132, processor 154 or processor 122). The processor may display the ultrasound image feed on a display device that is communicatively connected to the ultrasound scanner 102 (e.g., on screen 152 of display device 150). The displayed ultrasound image feed may include a ROI for imaging the out-of-plane needle.

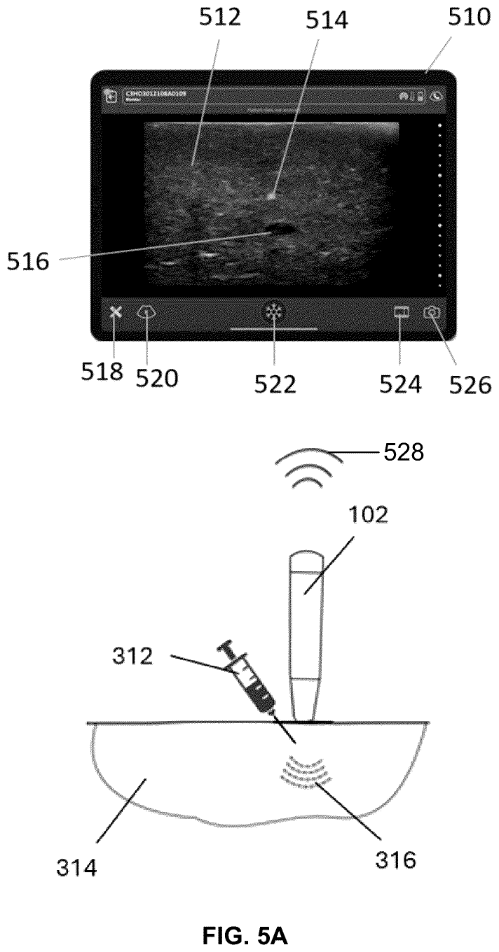

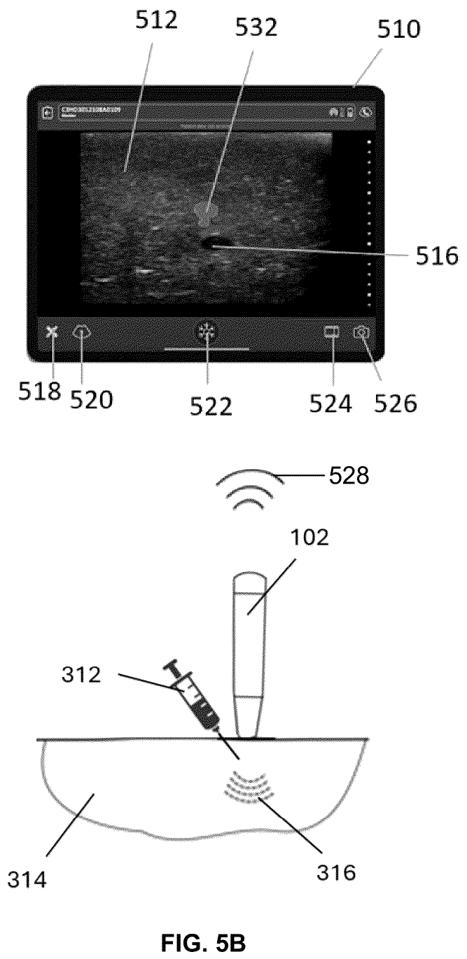

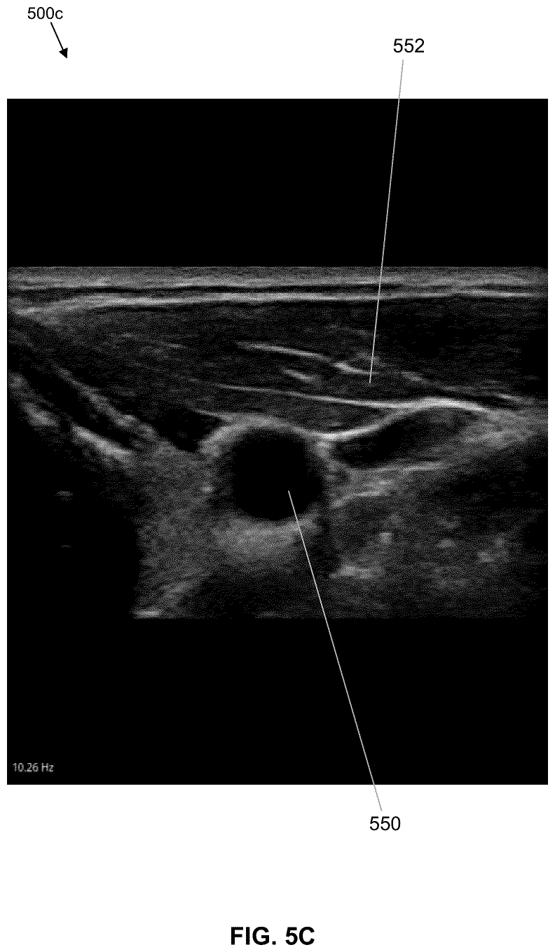

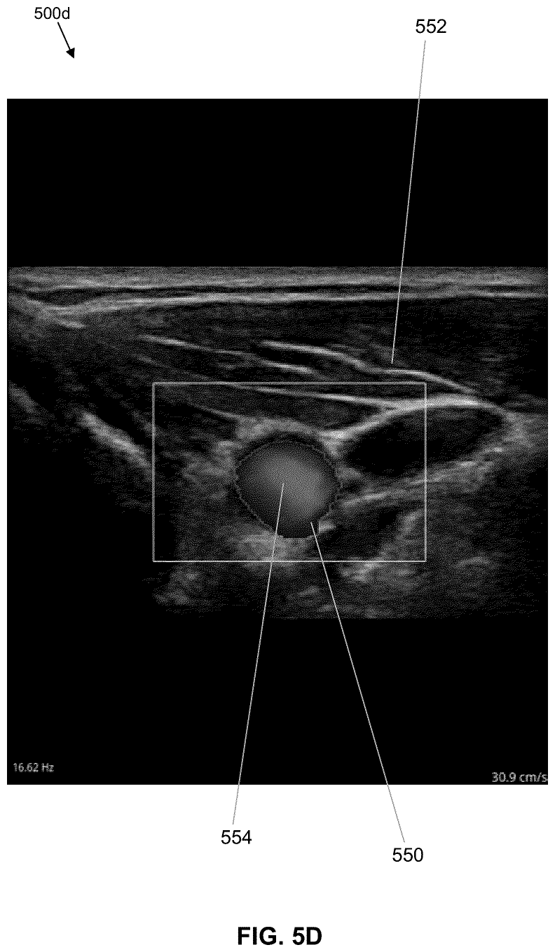

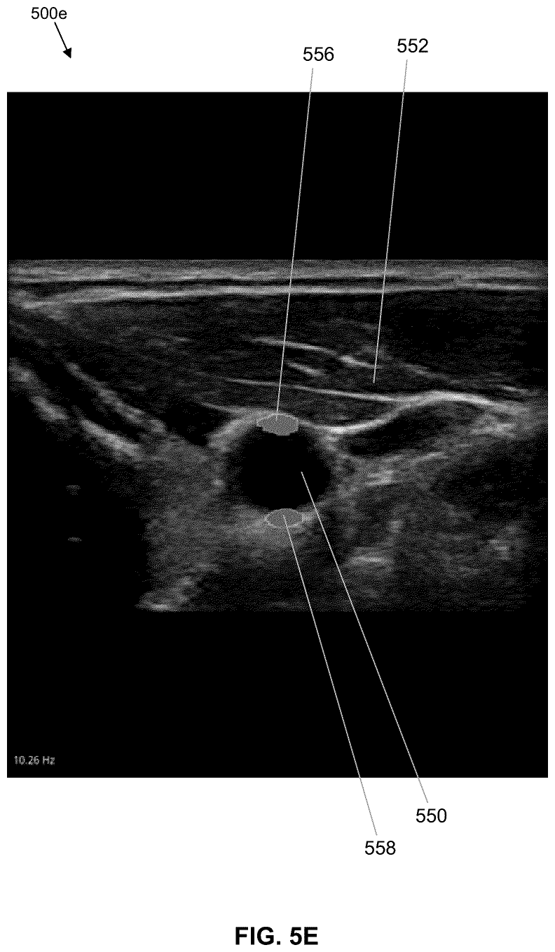

Reference is now also made to FIGS. 5A and 5B showing exemplary graphical user interface (GUI) 510 and GUI 530 respectively. GUIs 510 and 530 may be provided to a user based on ultrasound data 526 received from the ultrasound scanner 102 during usage of out-of-plane needle 312, e.g., as shown in FIG. 3B.

GUI 510 of FIG. 5A shows a B-mode ultrasound image of a region of interest 512 including a vessel 516 and an imaged needle 514 (corresponding to the out-of-plane needle 312) located above vessel 516. In the illustrated example, GUI 510 further includes a settings icon 518, a mode indicator 520, a freeze button 522, a video icon 524, and a camera icon 526 to enable the user to interact with the ultrasound image feed. As illustrated by FIG. 5A, it may be challenging for the user to track the out-of-plane needle 312 based on the imaged needle 514 in the displayed B-mode ultrasound image.

Method 400a includes generating a visual indicator onto the ultrasound image feed to address the above-described challenge and to enable the user to accurately track the out-of-plane needle. GUI 530 of FIG. 5B includes a visual indicator that may be generated at act 424 (as described herein below) of method 400a. Like GUI 510, GUI 530 shows the ultrasound image of the region of interest 512 including the vessel 516. In the illustrated example, GUI 530 also includes a settings icon 518, a mode indicator 520, a freeze button 522, a video icon 524, and a camera icon 526. GUI 530 further includes a visual indicator 532 that identifies the spatial location of the imaged out-of-plane needle within the region of interest (above the vessel 516 in the illustrated example). The visual indicator 532 can be generated based on a tissue Doppler mode ultrasound signal, as described herein below.

At act 414, the method 400a includes activating a tissue Doppler mode of the ultrasound scanner. In the tissue Doppler mode, the ultrasound scanner obtains a tissue Doppler ultrasound signal corresponding to the ROI. In some embodiments, the tissue Doppler mode may be activated in response to a user input. For example, the processor may activate the tissue Doppler mode of the ultrasound scanner 102 in response to user interactions provided via settings icon 518.

At act 422, the method 400a includes identifying a tissue velocity within the ROI while the out-of-plane needle is imaged within the region of interest. The tissue velocity can be associated with tissue movement caused by interactions with the out-of-plane needle. The processor may apply one or more image processing filters to the tissue Doppler ultrasound signal to measure the tissue velocity. The image processing filters may be applied to identify the low-frequency Doppler shifts originating from tissue movements caused by interaction with the out-of-plane needle.

In some embodiments, the method 400a may include act 420. At act 420, the method 400a includes adjusting at least one processing filter for the tissue Doppler ultrasound signal obtained at act 414. The processing filter may be applied to the tissue Doppler ultrasound signal obtained at act 414 and the filtered tissue Doppler ultrasound signal may be used to identify the tissue velocity at act 422.

For example, the processor may apply one or more temporal filters to preserve the tissue Doppler ultrasound signals. In some embodiments, this may be achieved by optimizing the persistence/frame averaging of Doppler mode images. Temporal resolution is described by a frame rate which is defined as the number of ultrasound images displayed in one second and is expressed in Hertz (Hz). High frame rates enable viewing of rapidly moving structures (such as heart valves) without motion artifacts, and also perform velocity and deformation analysis (i.e., tissue Doppler). Persistence refers to temporal smoothing used in both gray scale and color Doppler imaging. Successive frames are averaged as they are displayed to reduce the variations in the image between frames, hence lowering the temporal resolution of the image. Adjusting the image persistence causes individual frames of the scan to linger, thus blending them with the images in the successive frames. This causes incremental degrees of smoothing to the ultrasound image. Increasing persistence will smooth the image and reduce the frame rate; however, it can also create ghosting. The frame rate and the persistence can be optimized within the scope of the present invention to preserve the tissue Doppler ultrasound signal at a higher level compared with general diagnostic operations. Creating a preserved Doppler signal may enable a higher accuracy determination of a spatial location and an insertion/retraction direction of the out-of-plane needle in the subsequent acts of method 400a. As the end goal of the method and system of the invention is not an analysis of blood flow, an analysis of the condition of the vessels or other core diagnostic steps, optimizing either or both of persistence and frame rate to different levels than conventionally used, in order to create the preserved Doppler signal, may not be detrimental but may rather aid in determining: i) a more highly confirmed spatial location of the imaged needle within the region of interest, and ii) the insertion/retraction direction of the imaged needle.

There are a variety of techniques known and employed in the art to apply temporal filters to ultrasound images. Without limiting the generality of the foregoing, the teachings of the following are incorporated herein by reference: U.S. Pat. Publication 2014/0357999, U.S. Pat. Nos. 5,357,580, 8,721,549 and U.S. Pat. Publication 2012/0136252.

As another example of the processing filters at act 420, the processor may apply a wall filter to the obtained tissue Doppler ultrasound signal to reduce image artifacts resulting from wall motion of vessels within the region of interest. The wall filter may be configured to filter out all frequency shifts that fall below a selected threshold, with the intent of eliminating the lowest Doppler shifts that usually result from vessel (vascular) wall motion. These shifts are referred to as noise, clutter, or motion artifacts and are characterized by a low frequency and a high intensity/amplitude.

Reference is now also made to FIGS. 5C-5E. FIGS. 5C and 5E show exemplary ultrasound images 500c and 500e respectively that may be generated during implementation of method 400a. Image 500c shows a B-mode ultrasound image of a human anatomical structure 552 including a vessel 550. In FIG. 5D, image 500d illustrates a color Doppler mode image of the human anatomical structure 552 showing typical blood flow 554 through the vessel 550, without the additional application of tissue Doppler, as described in the implementation of the present invention, by way of comparison. In FIG. 5E, image 500e shows the application of tissue Doppler mode on an image of the human anatomical structure 552 showing image artifacts 556 and 558 that can result from wall motion of vessel 550.

The wall filter settings may be adjusted to distinguish between low-frequency Doppler shifts originating from slow-moving blood and those originating from tissue movement. The wall filter settings may be optimized to remove the low-frequency shifts associated with slow flow blood and vessel wall motion while preserving the low-frequency shifts associated with tissue movement. Within the scope of the invention, ultrasound scanners may comprise “auto-scan” control functions that automatically adjusts settings (including filter settings) according to a selected application.

There are a variety of techniques known and employed in the art to apply such wall filters to ultrasound images. Without limiting the generality of the foregoing, the teachings of the following are incorporated herein by reference: U.S. Pat. No. 6,760,486.

As another example of the processing filters at act 420, the processor may apply a spatial median filter to the obtained tissue Doppler ultrasound signal to improve the image resolution.

At act 424, the method 400a includes generating a visual indicator onto the ultrasound image feed to identify the imaged out-of-plane needle. The processor may generate the visual indicator to identify a spatial location of the imaged out-of-plane needle within the region of interest. The generated visual indicator may further identify one of an insertion direction and a retraction direction of the imaged out-of-plane needle within the region of interest. In some embodiments, the processor may automatically vary an opacity of the generated visual indicator to improve viewability of the underlying grayscale data.

Reference is now made to FIGS. 6A to 6J showing exemplary ultrasound images 600a-600j respectively that may be presented to a user via a graphical user interface (e.g., via GUI 510 of FIG. 5A or GUI 530 of FIG. 5B).

Image 600a shows a B-mode ultrasound image of an out-of-plane needle inserted above a vessel 612 in an ultrasound frame 614. Image 600a may be received and displayed, for example, at act 410 of the method 400a. As illustrated by FIG. 6A, it may be challenging for a user to accurately track the location and trajectory of the out-of-plane needle based on the imaged out-of-plane needle 610 in the B-mode ultrasound image.

Image 600b shows a tissue Doppler mode image including a visual indicator 620 identifying the out-of-plane needle inserted above the vessel 612 in the ultrasound frame 614. Image 600b may be displayed, for example, at act 424 of the method 400a. The position of the visual indicator 620 in the image 600b can identify the spatial location of the imaged out-of-plane needle. A color of the visual indicator 620 (e.g., the blue color in the illustrated example) can indicate that the out-of-plane needle is being inserted into a subject within a region of interest. The generated visual indicator 620 can enable the user to accurately track the location and trajectory of the out-of-plane needle within the region of interest.

As another example of the visual indicator generated at act 424 of the method 400a, image 600c shows a generated visual indicator 622 that identifies the out-of-plane needle being retracted from the ultrasound frame, within the region of interest 614. The position of the visual indicator 622 in the image 600c can identify the spatial location of the imaged out-of-plane needle. A color of the visual indicator 622 (e.g., the red color in the illustrated example) can indicate that the out-of-plane needle is being retracted from the ultrasound frame. The generated visual indicator 622 can enable the user to accurately track the location and trajectory of the out-of-plane needle within the region of interest.

As further examples of images displayed at acts 410 and 424 of the method 400a, image 600d shows a B-mode ultrasound image of an out-of-plane needle inserted into the vessel 612 in a region of interest within ultrasound frame 614. Image 600d may be displayed at act 410 of the method 400a. Image 600e shows a generated visual indicator 632 that identifies the out-of-plane needle being inserted into the vessel 612. The position of the visual indicator 632 in the image 600e can enable the user to identify the spatial location of the imaged out-of-plane needle within the vessel 612. A color of the visual indicator 632 (e.g., the blue color in the illustrated example) can indicate that the out-of-plane needle is being inserted into the vessel 612. The generated visual indicator 632 can enable the user to accurately track the location and trajectory of the out-of-plane needle within the region of interest. A brighter spot on the ultrasound image, 630, is an imaged out of plane needle, corresponding to needle 312.

FIG. 6F is illustrative of the placement, at image 600f, of a color box/sample box 640 within ultrasound image frame 614, captured by tissue Doppler mode, wherein a generated visual indicator 632 identifies an out-of-plane needle being inserted into the vessel 612.

FIG. 6G shows, at image 600g, the identification of a visual cue (illustrated as brighter spot 630 from FIG. 6D, colorized as 630a) on ultrasound image frame 614, representative of the movement of out of plane needle 312 in proximity to vessel 612.

The opacity of a visual cue or colorization can also be adjusted on a display screen, according to viewer/user preference, as shown generally as 600h in FIG. 6H, as 600i in FIG. 6I and as 600j in FIG. 6J. In FIG. 6H, at display screen interface 811, there is displayed image 614 comprising a region of interest comprising vessel 612 and on which an out of plane needle is sought to be detected. A selection of icons at the left of interface screen 811 includes an AI icon 808, image appearance icon 809 and overall gain icon 810. Activation of slider control 809 exposes a carousel wheel exposing icons for AI opacity 829, sector 828, dynamic range 826 and B gain 824. Adjustments to opacity are enabled through user activation of the AI opacity icon 829, activation of which exposes slider icon 850 on display screen 811. Adjustments to opacity may be made by directional movement of slider icon 850 in an increasing or decreasing direction. Opacity percentages are provided on display screen interface 811, with opacity decreasing to 26% in FIG. 6I (opacity shown as 818) and opacity increasing to 85% in FIG. 6J (opacity shown as 822). In this way, opacity is displayed on each of the images upon change. Display screen interface screen may additionally comprise one or more controls and guides which are not shown, including but not limited to an imaging mode selector, a freeze button, a video icon, a screen capture icon, a tools icon and depth indicator.

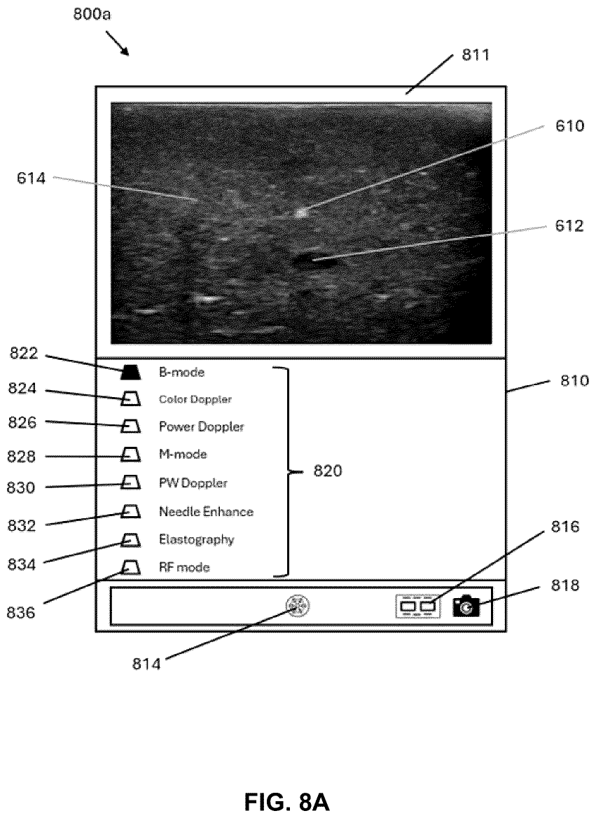

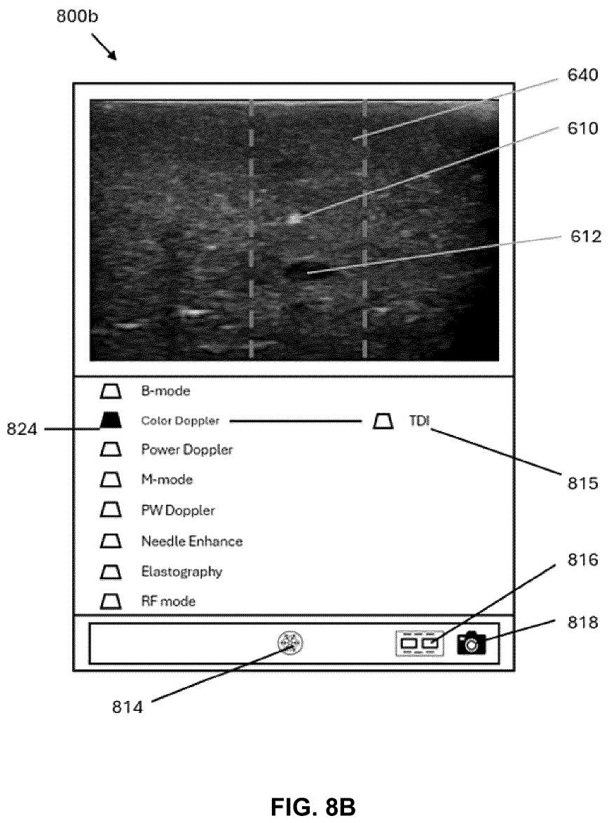

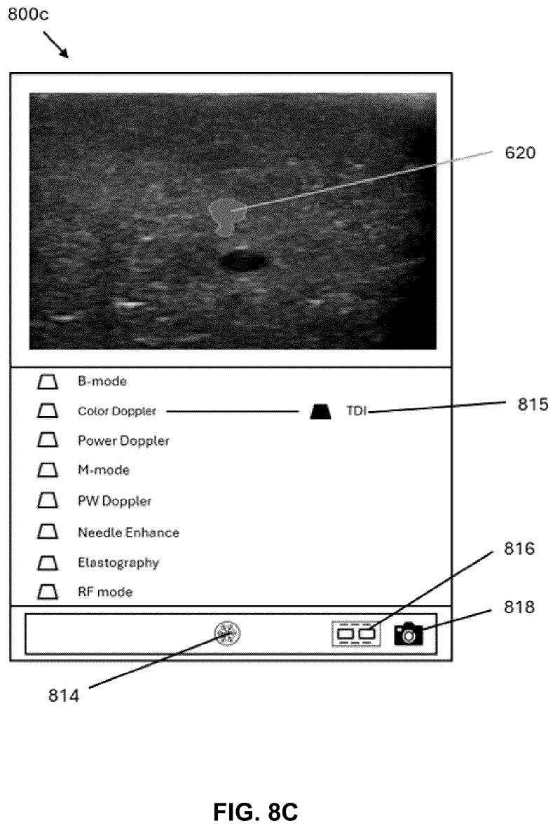

FIGS. 8A, 8B and 8C illustrate a series of sequential operational steps, performed via multi-use display device 810, communicably connected to an ultrasound imaging device 102, for the implementation of one embodiment of the present invention. FIG. 8A provides display screen interface 811 showing vessel 612 within image frame 614, and a bright area which is imaged out-of-plane needle 610. On display screen interface 811 are exemplary user experience/interaction features (collectively, 820) such as freeze screen contact point 814, film contact point 816, photo capture contact point 818, along with a drop-down menu comprising a plurality of options including: B-mode 822, Color Doppler 824, Power Doppler 826, M-Mode 828, PW Doppler 830, Needle Enhance 832, Elastography 834, and RF Mode 836. In FIG. 8A, an icon for B-mode is selected and images are acquired by the ultrasound imaging device in B-mode. In FIG. 8B, color Doppler is selected via color Doppler (icon) 824, and color box 640 is automatically placed. Selection of color Doppler exposes, on the interface, the option of tissue Doppler (TDI) at 815. In FIG. 8C, with TDI thus being activated, a tissue Doppler mode image including a visual indicator 620 identifying the out-of-plane needle inserted above the vessel 612 is displayed.

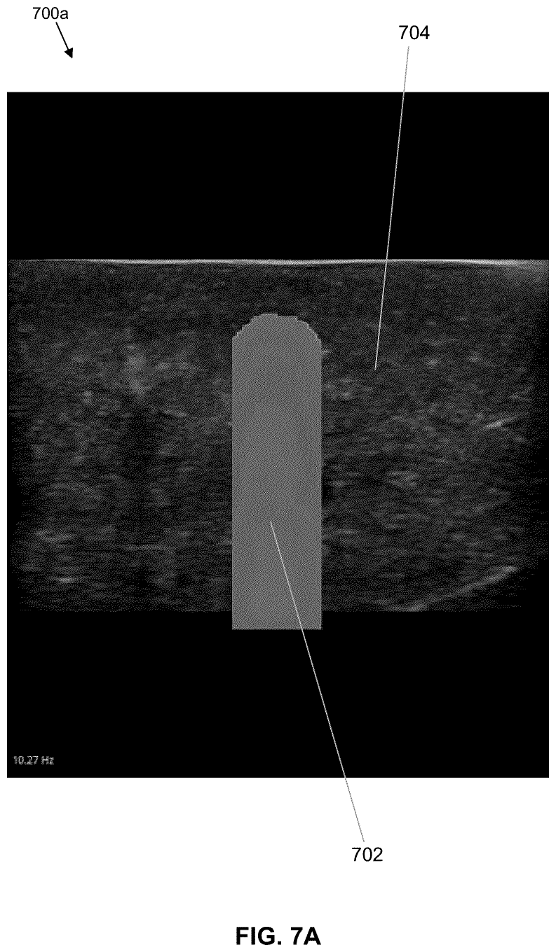

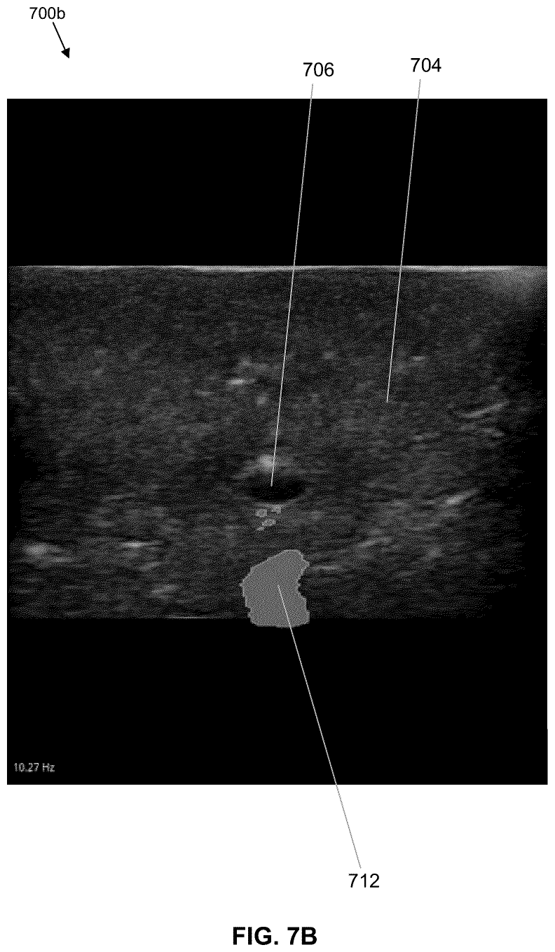

In some embodiments, one or more operational guidelines may be implemented for execution of method 400a. The operational guidelines may enable reduction in imaging artifacts generated during execution of method 400a. Reference is now made to FIGS. 7A and 7B showing exemplary ultrasound images 700a and 700b respectively that may be generated during execution of method 400a. Images 700a and 700b show image artefacts 702 and 712 respectively, that are generated at act 424 for an ultrasound frame within a region of interest 704 comprising a vessel 706. Image artefacts 702 and 712 may be caused by an up-and-down motion of the ultrasound scanner in a direction substantially parallel to the ultrasound beam. Image 700a corresponds to an example implementation of method 400a with no filtering applied to reduce motion artefacts, while image 700b corresponds to an example implementation of method 400a with filtering applied to reduce motion artefacts. The one or more operational guidelines may be implemented by a user to reduce the up-and-down motion to reduce the motion artefacts while the tissue Doppler mode is activated.

Reference will now be made to FIG. 4B. FIG. 4B is a flowchart diagram showing acts of a method 400b. Like the method 400a of FIG. 4A, the method 400b may be implemented for tracking an out-of-plane needle within an ultrasound image feed that is acquired from an ultrasound scanner. Method 400b may generally be performed using any suitable ultrasound imaging system. For example, method 400b may be performed using ultrasound imaging system 100 shown in FIG. 1 or ultrasound imaging system 200 shown in FIG. 2 and concurrent reference is made herein below to the system components shown in FIGS. 1 and 2.

At act 410, the method 400b (like method 400a) includes receiving and displaying, on a display device, one or more B-mode images of a region of interest for imaging the out-of-plane needle. For example, ultrasound scanner 102 may acquire a plurality of new ultrasound image frames and provide the acquired ultrasound image feed to a processor (e.g., processor 132, processor 154 or processor 122). The processor may display the ultrasound image feed on a display device that is communicatively connected to the ultrasound scanner 102 (e.g., on screen 152 of display device 150). The displayed ultrasound image feed may include a ROI for imaging the out-of-plane needle.

In some embodiments, the method 400b may include act 412. In other embodiments, the method 400b may not include act 412 and the method 400b may proceed directly from act 410 to act 414.

At act 412, the method 400b includes optimizing the B-mode image received at act 410. One or more filters may be applied to preserve the features of the B-mode images, prior to switching to a tissue Doppler mode at act 414. For example, this can be achieved by reducing the noise levels (for example, Salt and Pepper Noise (impulse or spike noise), Poisson noise (shot noise), Gaussian or amplifier noise and Speckle Noise). This reduction may be achieved by use of one or more of the following non-limiting filter types: Gaussian filter, bilateral filter, Order statistic filter, Mean filter and Laplacian filter.

As such, within the scope of the invention, additional basic parameters for the B-mode (grayscale) examination may preferably be optimized, not only for higher-quality images but also to facilitate the subsequent tissue Doppler mode component of the method. These basic parameters may comprise (a) the location and number of focal zones, (b) the depth of field for the ROI being imaged, (c) the two-dimensional (2D) gain setting, (d) the scan orientation, (c) the image zoom settings, and, where possible and depending on the equipment being used, (f) the presets for the specific transducer being used and the type of study being performed. Because the method 400b may include superimposing color Doppler data on the 2D image, a high 2D gain setting suppresses color information and a low setting highlights color information. The frame rate varies inversely with the depth of field: sampling from a deeper segment slows the frame rate.

In some embodiments, at act 412, the method 400b includes applying at least one signal processing filter to the B-mode images, prior to switching to the tissue Doppler mode. For example, the signal processing filter may include a wall filter to remove/reduce flash artifacts.

At act 414, the method 400b (like method 400a) includes activating a tissue Doppler mode of the ultrasound scanner. In the tissue Doppler mode, the ultrasound scanner obtains a tissue Doppler ultrasound signal corresponding to the ROI. In some embodiments, the tissue Doppler mode may be activated in response to a user input. For example, the processor may activate the tissue Doppler mode of the ultrasound scanner 102 in response to user interactions provided via settings icon 518.

In some embodiments, the method 400b may include act 416. In other embodiments, the method 400b may not include act 416 and the method 400b may proceed directly from act 414 to act 418.

At act 416, the method 400b includes applying a color box to the region of interest. The color box may be placed over the ROI, manually or optimally using an artificial intelligence model. Without limiting the generality of the foregoing, the teachings of the following are incorporated herein by reference for AI placement of a color box: U.S. Pat. Publication 2022/0061810. The processor may be configured to select the most prominent tissue Doppler ultrasound signal from the ROI within the color box for further processing.

At act 418, the method 400b includes applying one or more temporal filters to the tissue Doppler ultrasound signal to optimize the persistence/frame averaging of Doppler mode images. As described herein above with reference to act 420 of method 400a (FIG. 4A), the frame rate and the persistence can be optimized within the scope of the present invention to preserve the tissue Doppler ultrasound signal at a higher level compared with general diagnostic operations. This can enable a higher accuracy determination of a spatial location and an insertion/retraction direction of the out-of-plane needle in the subsequent acts of method 400b.

At act 422, the method 400b (like method 400a) includes identifying a tissue velocity within the ROI while the out-of-plane needle is imaged within the region of interest. The tissue velocity can be associated with tissue movement caused by interactions with the out-of-plane needle.

At act 424, the method 400b (like method 400a) includes generating a visual indicator onto the ultrasound image feed to identify the imaged out-of-plane needle. The processor may generate the visual indicator to identify a spatial location of the imaged out-of-plane needle within the region of interest. The generated visual indicator may further identify one of an insertion direction and a retraction direction of the imaged out-of-plane needle within the region of interest.

It is intended that methods and systems of the present invention have wide application in a variety of therapies, procedures, and treatments. Without limiting the generality of the foregoing, the methods and systems of the present invention may be used in intervention procedures (e.g., nerve blocks, vascular access), wherein needles are used for administration of medicine or evacuation of fluid contents. In the case of nerve blocks it is desirous to avoid all vascular vessels even though many nerves are very closely associated with blood vessels. The methods and systems of the present invention may be used in a variety of aesthetic and cosmetic procedures that are based around injectables wherein avoiding vascular vessels can be a matter of life and death. The methods and systems of the present invention may be used in surgical procedures such as the Brazilian Butt Lift (BBL) wherein fat is injected into the gluteus region and avoiding vascular features is essential to avoid fat being erroneously deposited therein. The methods and systems of the present invention provide a means for vascular vessel avoidance, without the need for a user to be limited to in-plane needle usage.

### C. Claim Support

In a first broad aspect of the present disclosure, there is provided a method for tracking an out-of-plane needle within an ultrasound image feed that is acquired from an ultrasound scanner, the method comprising: displaying, on a screen that is communicatively connected to the ultrasound scanner, the ultrasound image feed comprising a region of interest for imaging the out-of-plane needle; activating a tissue Doppler mode of the ultrasound scanner, in which the ultrasound scanner obtains a tissue Doppler ultrasound signal corresponding to the region of interest; identifying a tissue velocity within the region of interest while the out-of-plane needle is imaged within the region of interest; and generating a visual indicator onto the ultrasound image feed to identify the imaged out-of-plane needle.

In some embodiments, generating the visual indicator onto the ultrasound image feed to identify the imaged out-of-plane needle includes generating the visual indicator to identify a spatial location of the imaged out-of-plane needle within the region of interest.

In some embodiments, generating the visual indicator onto the ultrasound image feed to identify the out-of-plane needle includes generating the visual indicator to identify one of an insertion direction and a retraction direction of the imaged out-of-plane needle within the region of interest.

In some embodiments, the method additionally includes applying at least one image processing filter to the tissue Doppler ultrasound signal to measure the tissue velocity within the region of interest which includes: applying the at least one image processing filter to identify Doppler shift signals associated with tissue movement caused by interaction with the out-of-plane needle.

In some embodiments, the method additionally includes applying a temporal filter to the tissue Doppler ultrasound signal.

In some embodiments, the temporal filter includes an adaptive persistence filter for measuring the tissue velocity.

In some embodiments, the method additionally includes applying a spatial median filter to the tissue Doppler ultrasound signal to improve image resolution.

In some embodiments, the method additionally includes applying at least one image processing filter to the tissue Doppler ultrasound signal to measure the tissue velocity within the region of interest which includes: applying the at least one image processing filter to the tissue Doppler ultrasound signal to reduce image artifacts resulting from wall motion of vessels within the region of interest.

In some embodiments, the ultrasound image feed includes ultrasound image frames of the region of interest and the method further includes applying at least one signal processing filter to the ultrasound image frames prior to activation of the tissue Doppler mode.

In some embodiments, the at least one signal processing filter is a wall filter.

In some embodiments, generating the visual indicator onto the ultrasound image feed to identify the imaged out-of-plane needle includes automatically varying an opacity of the visual indicator.

In a second broad aspect of the present disclosure, there is provided an ultrasound imaging system for tracking an out-of-plane needle within an ultrasound image feed, comprising: an ultrasound scanner configured to acquire a plurality of new ultrasound image frames; a processor that is communicatively connected to the ultrasound scanner and configured to: display, on a screen that is communicatively connected to the ultrasound scanner, the ultrasound image feed comprising a region of interest for imaging the out-of-plane needle; activate a tissue Doppler mode of the ultrasound scanner, in which the ultrasound scanner obtains a tissue Doppler ultrasound signal corresponding to the region of interest; identify a tissue velocity within the region of interest while the out-of-plane needle is imaged within the region of interest; and generate a visual indicator on the ultrasound image feed to identify the imaged out-of-plane needle; and a display device configured to display at least the visual indicator to a system user.

In some embodiments, the visual indicator identifies a spatial location of the imaged out-of-plane needle within the region of interest.

In some embodiments, the visual indicator identifies one of an insertion direction and a retraction direction of the imaged out-of-plane needle within the region of interest.

In some embodiments, after activating the tissue Doppler mode, the processor applies at least one image processing filter to the tissue Doppler ultrasound signal to measure the tissue velocity within the region of interest which includes applying the at least one image processing filter to identify Doppler shift signals associated with tissue movement caused by interaction with the out-of-plane needle.

In some embodiments, the at least one image processing filter includes a temporal filter.

In some embodiments, the processor applies at least one image processing filter to the tissue Doppler ultrasound signal to measure the tissue velocity within the region of interest which includes: applying the at least one image processing filter to the tissue Doppler ultrasound signal to reduce image artifacts resulting from wall motion of vessels within the region of interest.

In some embodiments, the processor applies at least one signal processing filter to the ultrasound image frames prior to activation of the tissue Doppler mode.

In some embodiments, the at least one signal processing filter is a wall filter.

In a third broad aspect of the present disclosure, there is provided a computer readable medium storing instructions for execution by a processor communicatively coupled with an ultrasound scanner, within an ultrasound imaging system, wherein when the instructions are executed by the processor, it is configured to: display, on a screen that is communicatively connected to the ultrasound scanner, the ultrasound image feed comprising a region of interest for imaging the out-of-plane needle; activate a tissue Doppler mode of the ultrasound scanner, in which the ultrasound scanner obtains a tissue Doppler ultrasound signal corresponding to the region of interest; identify a tissue velocity within the region of interest while the out-of-plane needle is imaged within the region of interest; and generate a visual indicator on the ultrasound image feed to identify the imaged out-of-plane needle.

### D. Interpretation of Terms

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms.

Unless the context clearly requires otherwise, throughout the description and the claims:

Words that indicate directions such as “vertical”, “transverse”, “horizontal”, “upward”, “downward”, “forward”, “backward”, “inward”, “outward”, “vertical”, “transverse”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Embodiments of the invention may be implemented using specifically designed hardware, configurable hardware, programmable data processors configured by the provision of software (which may optionally comprise “firmware”) capable of executing on the data processors, special purpose computers or data processors that are specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”). Examples of programmable data processors are: microprocessors, digital signal processors (“DSPs”), embedded processors, graphics processors, math co-processors, general purpose computers, server computers, cloud computers, mainframe computers, computer workstations, and the like. For example, one or more data processors in a control circuit for a device may implement methods as described herein by executing software instructions in a program memory accessible to the processors.

For example, while processes or blocks are presented in a given order herein, alternative examples may perform routines having steps, or employ systems having blocks, in a different order, and some processes or blocks may be deleted, moved, added, subdivided, combined, and/or modified to provide alternative or sub combinations. Each of these processes or blocks may be implemented in a variety of different ways. Also, while processes or blocks are at times shown as being performed in series, these processes or blocks may instead be performed in parallel or may be performed at different times.

The invention may also be provided in the form of a program product. The program product may comprise any non-transitory medium which carries a set of computer-readable instructions which, when executed by a data processor (e.g., in a controller and/or ultrasound processor in an ultrasound machine), cause the data processor to execute a method of the invention. Program products according to the invention may be in any of a wide variety of forms. The program product may comprise, for example, non-transitory media such as magnetic data storage media including floppy diskettes, hard disk drives, optical data storage media including CD ROMs, DVDs, electronic data storage media including ROMs, flash RAM, EPROMs, hardwired or preprogrammed chips (e.g., EEPROM semiconductor chips), nanotechnology memory, or the like. The computer-readable signals on the program product may optionally be compressed or encrypted.

Where a component (e.g. a software module, processor, assembly, device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

To aid the Patent Office and any readers of any patent issued on this application in interpreting the claims appended hereto, applicant wishes to note that they do not intend any of the appended claims or claim elements to invoke 35 U.S.C. 112 (f) unless the words “means for” or “step for” are explicitly used in the particular claim.

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples but should be given the broadest interpretation consistent with the description as a whole.

1. A method for tracking an out-of-plane needle within an ultrasound image feed that is acquired from an ultrasound scanner, the method comprising:
displaying, on a screen that is communicatively connected to the ultrasound scanner, the ultrasound image feed comprising a region of interest for imaging the out-of-plane needle;
activating a tissue Doppler mode of the ultrasound scanner, in which the ultrasound scanner obtains a tissue Doppler ultrasound signal corresponding to the region of interest;
identifying a tissue velocity within the region of interest while the out-of-plane needle is imaged within the region of interest; and
generating a visual indicator onto the ultrasound image feed to identify the imaged out-of-plane needle.

2. The method of claim 1, wherein generating the visual indicator onto the ultrasound image feed to identify the imaged out-of-plane needle comprises generating the visual indicator to identify a spatial location of the imaged out-of-plane needle within the region of interest.

3. The method of claim 1, wherein generating the visual indicator onto the ultrasound image feed to identify the out-of-plane needle comprises generating the visual indicator to identify one of an insertion direction and a retraction direction of the imaged out-of-plane needle within the region of interest.

4. The method of claim 1, additionally comprising applying at least one image processing filter to the tissue Doppler ultrasound signal to measure the tissue velocity within the region of interest which comprises:
applying the at least one image processing filter to identify Doppler shift signals associated with tissue movement caused by interaction with the out-of-plane needle.

5. The method of claim 1, additionally comprising applying a temporal filter to the tissue Doppler ultrasound signal.

6. The method of claim 5, wherein the temporal filter comprises an adaptive persistence filter for measuring the tissue velocity.

7. The method of claim 1, additionally comprising applying a spatial median filter to the tissue Doppler ultrasound signal to improve image resolution.

8. The method of claim 1, additionally comprising applying at least one image processing filter to the tissue Doppler ultrasound signal to measure the tissue velocity within the region of interest which comprises:
applying the at least one image processing filter to the tissue Doppler ultrasound signal to reduce image artifacts resulting from wall motion of vessels within the region of interest.

9. The method of claim 1, wherein the ultrasound image feed comprises ultrasound image frames of the region of interest and the method further comprises applying at least one signal processing filter to the ultrasound image frames prior to activation of the tissue Doppler mode.

10. The method of claim 9 wherein the at least one signal processing filter is a wall filter.

11. The method of claim 1 wherein generating the visual indicator onto the ultrasound image feed to identify the imaged out-of-plane needle comprises automatically varying an opacity of the visual indicator.

12. An ultrasound imaging system for tracking an out-of-plane needle within an ultrasound image feed, comprising:
an ultrasound scanner configured to acquire a plurality of new ultrasound image frames;
a processor that is communicatively connected to the ultrasound scanner and configured to:
a. display, on a screen that is communicatively connected to the ultrasound scanner, the ultrasound image feed comprising a region of interest for imaging the out-of-plane needle;
b. activate a tissue Doppler mode of the ultrasound scanner, in which the ultrasound scanner obtains a tissue Doppler ultrasound signal corresponding to the region of interest;
c. identify a tissue velocity within the region of interest while the out-of-plane needle is imaged within the region of interest; and
d. generate a visual indicator on the ultrasound image feed to identify the imaged out-of-plane needle; and
a display device configured to display at least the visual indicator to a system user.

13. The system of claim 12 wherein the visual indicator identifies a spatial location of the imaged out-of-plane needle within the region of interest.

14. The system of claim 12 wherein the visual indicator identifies one of an insertion direction and a retraction direction of the imaged out-of-plane needle within the region of interest.

15. The system of claim 12 wherein, after activating the tissue Doppler mode, the processor applies at least one image processing filter to the tissue Doppler ultrasound signal to measure the tissue velocity within the region of interest which comprises applying the at least one image processing filter to identify Doppler shift signals associated with tissue movement caused by interaction with the out-of-plane needle.

16. The system of claim 15 wherein the at least one image processing filter comprises a temporal filter.

17. The system of claim 12 wherein the processor applies at least one image processing filter to the tissue Doppler ultrasound signal to measure the tissue velocity within the region of interest which comprises:
applying the at least one image processing filter to the tissue Doppler ultrasound signal to reduce image artifacts resulting from wall motion of vessels within the region of interest.

18. The system of claim 12 wherein the processor applies at least one signal processing filter to the ultrasound image frames prior to activation of the tissue Doppler mode.

19. The system of claim 18 wherein the at least one signal processing filter is a wall filter.

20. A computer readable medium storing instructions for execution by a processor communicatively coupled with an ultrasound scanner, within an ultrasound imaging system, wherein when the instructions are executed by the processor, it is configured to:
a. display, on a screen that is communicatively connected to the ultrasound scanner, the ultrasound image feed comprising a region of interest for imaging the out-of-plane needle;
b. activate a tissue Doppler mode of the ultrasound scanner, in which the ultrasound scanner obtains a tissue Doppler ultrasound signal corresponding to the region of interest;
c. identify a tissue velocity within the region of interest while the out-of-plane needle is imaged within the region of interest; and
d. generate a visual indicator on the ultrasound image feed to identify the imaged out-of-plane needle.
